(19) United States
(12) Patent Application Publication
(10) Pub. No.: US 2006/0179499 A1
(43) Pub. Date: Aug. 10, 2006

(54) Non-human animal models for diabetic complications and their uses

(75) Inventors: Rebecca S. Tirabassi, Sturbridge, MA (US); Edward H. Kislauskis, Medway, MA (US); Joan F. Flanagan, Worcester, MA (US); Dennis L. Guberski, Rutland, MA (US)

Correspondence Address: FISH & NEAVE IP GROUP;ROPES & GRAY LLP, ONE INTERNATIONAL PLACE, BOSTON, MA, 02110-2624, US

(73) Assignee: Biomedical Research Models, Inc.

(21) Appl. No.: 11/207,315

(22) Filed: Aug. 19, 2005

Related U.S. Application Data
(60) Provisional application No. 60/603,412, filed on Aug. 20, 2004.

(51) Int. Cl. A01K 67/027

(52) U.S. Cl. 800/14

(57) ABSTRACT

The instant invention relates to methods for generating a non-human animal model for a diabetic complication. The invention further relates to screening methods for therapeutics of diabetic complications using the animal model generated by the methods of the invention.

### REFERENCE TO RELATED APPLICATION

This application claims the benefit of the filing date of U.S. Provisional Application Ser. No. 60/603,412, entitled “NON-HUMAN ANIMAL MODELS FOR DIABETIC COMPLICATIONS AND THEIR USES,” and filed on Aug. 20, 2004. The teachings of the referenced application are incorporated herein by reference.

### GOVERNMENT FUNDING

Work described herein was funded, in whole or in part, by Grant No. 1R43-DK53679 and 2R44-DK53679 from the National Institute of Diabetes and Digestive and Kidney Diseases. The United States government has certain rights in the invention.

### BACKGROUND OF THE INVENTION

Diabetes is a growing epidemic in the United States; it affects more than 6% of the US population. Diabetes and its associated complications present a significant healthcare burden. The annual medical cost for care of diabetic patients is more than $92 billion. These costs include direct costs for treatment of diabetes as well as $24.6 billion attributed to the care of chronic diabetic complications.

Diabetes causes a variety of physiological and anatomical irregularities, the most prominent of which is the inability of the body to utilize glucose normally, which results in hyperglycemia. Chronic diabetes can lead to complications of the vascular system which include abnormalities involving large and medium size blood vessels (macrovascular diseases) and abnormalities involving small blood vessels such as arterioles and capillaries (microvascular diseases). The thickening and leakage of capillaries caused by diabetes primarily affect the eyes (retinopathy) and kidneys (nephropathy). The thickening and leakage of capillaries caused by diabetes are also associated with skin disorders, disorders of the nervous system (neuropathy) and impotence. The eye diseases associated with diabetes are nonproliferative diabetic retinopathy, proliferative diabetic retinopathy, diabetic maculopathy, glaucoma and cataracts. It is estimated that up to 50% of diabetics will develop diabetic nephropathy, and ultimately renal failure, between 10 and 30 years from the time of onset of the diabetes. Diabetic neuropathy is the most common microvascular complication and can present as several syndromes that affect motor, sensory and autonomic nerves. Erectile dysfunction is also a frequent occurrence in diabetic male patients.

Diabetic complications significantly shorten and impair the quality of life of diabetic patients. It would be desirable to have effective treatment methods for diabetic complications.

Current candidate therapeutics aimed at treating diabetic complications have to date proved disappointing in clinical trials, despite their initial promises in animal models. The study of diabetic complications in available animal models is currently cost-prohibitive. Animals must be aged to greater than 6 months of age and enormous care must be taken to manage the diabetic state. It would therefore also be desirable to have an non-human animal model that approximates human diabetic complications with an earlier onset and/or greater severity than currently available animal models.

### SUMMARY OF THE INVENTION

The invention is based, at least in part, on Applicants' discovery that the ligation or cross-linking of Toll-Like Receptors (TLRs) may initiate a self-perpetuating inflammatory process in diabetic patients as a result of high glucose concentrations. Once initiated, this inflammatory process is maintained even when glucose homeostasis returns to normal. Building on this important discovery, the present invention features methods for treating or preventing a diabetic complication by administering to an individual an agent that interferes with a TLR signaling cascade. The agent, such as, for example, a TLR antibody may interfere directly with a TLR. The agent may also interfere indirectly with a TLR by acting on a component upstream or downstream of TLR in the TLR signaling pathway.

The present invention also features methods for generating non-human animal models for diabetic complications that enable more cost-effective studies of the diabetic complications. In the methods, a non-human animal is administered a TLR agonist in an amount sufficient to induce at least one (a, one or more) diabetic complication. Animal models of ths invention have at least one diabetic complication with an earlier onset, a higher incidence, and/or greater severity compared to currently available diabetic animal models. Preferably, the animal model is a rodent model, e.g., a rat model. The diabetic complications include, for example, neuropathy, nephropathy, retinopathy, peripheral circulation disorders, erectile dysfunction in male diabetic patients, and skin ulcerations. The present invention additionally provides methods of screening for therapeutic agents useful for treating or preventing a diabetic complication or complications.

Thus one aspect of the invention provides a method of providing a non-human animal model for at least one diabetic complication, the method comprising administering to the non-human animal a Toll-Like Receptor (TLR) agonist in an amount sufficient to induce said at least one diabetic complication in the animal.

The TLR agonist may be an agonist for TLR3.

The animal may be a rodent, such as a rat, a mouse, a hamster, a guinea pig, etc. Or the animal may be other laboratory, farm animals (cattle, horse, pig, sheep, goat, etc.), pets (e.g., cat or dog, etc.), or non-human primates.

In certain embodiments, the rat may be a biobreeding Zucker diabetic rat (BBZDR/Wor).

The diabetic complication may manifest in the animal at least about 1 month, 2 months, 3 months, 4 months, 5 months, 6 months or >6 months earlier than that in an available rat model, such as those animal models selected from: Streptozotocin-induced diabetic rat, biobreeding diabetes prone rat (BBDP/Wor), biobreeding diabetes resistant rat (BBDR/Wor) or biobreeding Zucker diabetic rat (BBZDR/Wor).

The diabetic complication may manifest in the animal at about 1 month, 2 months, 3 months, 4 months, 5 month, or 6 months after the administration of the TLR agonist.

The diabetic complication may be a macrovascular complication or a microvascular complication, such as is neuropathy, retinopathy or nephropathy.

The TLR agonist may be an agonist for TLR2, TLR3, TLR4, TLR7, TLR9, or TLR11.

The animal model may develop the at least one diabetic complication in the absence of severe hyperglycemia and/or glycosuria.

The subject method may comprise administering to the non-human animal two or more Toll-Like Receptor (TLR) agonists.

Another aspect of the invention provides a method of screening for a therapeutic agent useful for treating or preventing a diabetic complication, comprising: (a) providing, by the method of the invention, a test animal and a substantially identical control animal; (b) administering a candidate agent to the test animal; (c) maintaining the test animal and the control animal under conditions appropriate for development of at least one diabetic complication in the control animal; (d) assessing said at least one diabetic complication in the test animal and the control animal; and, (e) comparing the severity and/or onset of the diabetic complication in the test animal with that of the control animal, wherein reduced severity and/or delay in the onset of the diabetic complication in the test animal indicates that the candidate agent is the therapeutic agent useful for treating or preventing the diabetic complication.

The the test animal and the control animal may be littermates.

The candidate agent may be a TLR antagonist.

Another aspect of the invention provides a method for treating, preventing, reversing or limiting the severity of a diabetic complication in an individual in need thereof, comprising administering to the individual an agent that interferes with TLR signaling, in an amount sufficient to interfere with TLR signaling.

Another aspect of the invention provides a method of treating, preventing, reversing or limiting the severity of a diabetic complication in an individual in need thereof, comprising administering to the individual an agent that interferes with at least one TLR signaling cascade.

The embodiments described above, including those described under different aspects of the invention, are contemplated to be applicable for all aspects of the inventions wherever appropriate.

### BRIEF DESCRIPTION OF DRAWINGS

FIG. 1 shows four alternative metabolic pathways resulting from high intracellular glucose concentration. The normal glycolysis pathway is shown by dotted arrows. High superoxide production inhibits the final conversion step of the pathway. This results in a build-up of intermediates in the pathway denoted by solid arrows. Each of the intermediates is then shunted into 4 alternative metabolic pathway: the polyol, hexosamine, diacylglycerol and advanced glycation endproducts pathways.

FIG. 2 shows an outline of the TLR experiments.

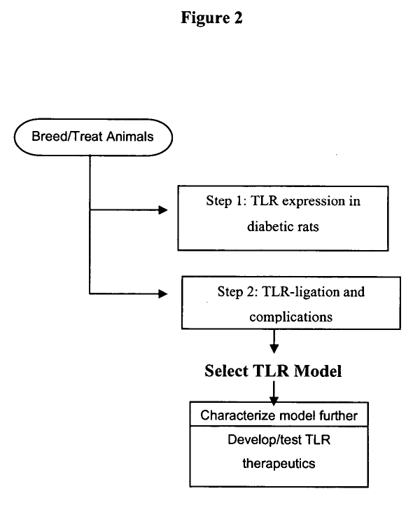

FIG. 3 shows an outline of animal maintenance for TLR expression studies. A total of 504 rats (168 of each genotype) are entered into the study. At 20-25 days of age, the animals are treated either with vehicle, streptozotocin (STZ), Kilham's rat virus (KRV), rat cytomegalovirus (RCMV), a TLR3 agonist (poly I:C), a TLR4 agonist (LP S) or the TLR2/6 agonist, zymosan. At the indicated ages (duration of diabetes shown in parantheses), 6 animals from each group are tested for motor nerve conduction velocities prior to being sacrificed. The indicated organs are harvested and processed for immunohistochemistry. Serum is analyzed by FACs.

FIGS. 4A-4F show BBZDR/Wor pancreatic islet morphology. Pancreata from lean (FIGS. 4A and 4B), obese prediabetic (FIGS. 4C and 4D), and obese diabetic (FIGS. 4E and 4F) BBZDR/Wor rats were isolated and processed for immunohistochemistry. Consecutive, fixed hemotoxylin and eosin-stained sections were immunostained for glucagon (FIGS. 4A, 4C, and 4E) or insulin (FIGS. 4B, 4D, and 4F).

### DETAILED DESCRIPTION OF THE INVENTION

I. Overview

As described in detail in the Exemplification section, Applicants have made the important recognition that (1) damages caused by physiological changes in diabetic individuals could be initiated through ligation of TLRs, and (2) ligation of TLRs initiates a self-perpetuating inflammatory process that is maintained even upon return to normal glucose homeostasis. Based on this recognition, the development of complications could be affected through regulating one or more TLR signaling cascades.

Accordingly, the present invention provides methods for treating or preventing a diabetic complication by administering to an individual an agent that interferes with TLR signaling, such as a TLR signaling cascade. In a diabetic individual who has a diabetic complication or complications (at least one complication), the agent, by interfering with TLR signaling, partially or completely turns off the self-perpetuating inflammatory process that sustains diabetic complications and, as a result, reduces the extent to which complications occur, prevents their further development, or reverses complications already initiated. In a diabetic individual in whom diabetic complications are not yet evident, the agent, by interfering with TLR signaling, prevents the onset of one or more diabetic complications, or reduces the extent to which they occur.

The present invention further provides methods for generating non-human animal models of diabetic complications by administering to the animal TLR agonists singly or in combination in an amount sufficient to induce the diabetic complication. In certain embodiments, the animals generated by the methods of the invention will develop diabetic complications with an earlier onset, a higher incidence and/or greater severity than currently available animal models for diabetic complications. Currently, studying diabetic complications in available animal models is cost prohibitive, largely due to the 6-8 months of animal care required before the animals develop diabetic complications. Hence, reducing the length of time required before the onset of a diabetic complication will directly translate into reduced costs of studying the diabetic complication. Some currently-available models of diabetic complications rely on the use of chemical agents such as streptozotocin to induce beta-cell damage in the absence of stimulation of inflammatory processes. Lack of inflammation limits the utility of these models as they do not accurately reflect human disease. Furthermore, the methods of the invention may generate animal models for a specific diabetic complication in the absence of other complications, and thus serve as a better model for studying that particular complication. The animals generated by the methods of the invention will allow faster and more cost-effective screening of therapeutic agents for diabetic complications. The animals generated by the methods of the invention are also useful for facilitating faster and more cost-effective validation of lead compounds generated from in vitro studies.

II. Toll-Like Receptors

TLRs are generally described as pattern recognition molecules that recognize foreign constituents (polysaccharides, proteins and nucleic acid patterns) expressed by invading pathogens. As such, TLRs are the immune system's first line of innate immune defense, recognizing and responding to newly encountered microbes without a need for prior exposure. Initial triggering of TLR signaling results in stimulation of inflammatory responses and induction of pathogen defense genes. Zhang et al., Science 303: 1522-1526, 2004. TLRs are also important in bridging innate and adaptive immune responses. TLR signaling stimulates the development of memory (adaptive) immune responses and molds the type of ensuing response. In addition to recognizing patterns associated with invading pathogens, TLRs also participate in “sterile inflammation,” recognizing aberrant expression of endogenous molecules that could signal ongoing pathology.

a. Structure.

Toll was first discovered as a transmembrane receptor required for appropriate dorso-ventral patterning during embryogenesis of Drosophila melanogaster and was subsequently found to be involved in innate immunity to fungal infections. Takeda et al., Annu. Rev. Immunol. 21: 335-376, 2003. It has since been recognized that Toll is a member of a large family of evolutionarily-conserved proteins involved in innate immune responses (Toll/IL-1 receptor family). The first mammalian Toll homolog, called a Toll-like receptor (TLR), was discovered approximately seven years ago. Since that time, 11 novel mammalian TLRs have been identified.

TLRs and IL-I receptors (IL-1Rs) are type 1 integral membrane proteins. While the extracellular domains of the proteins are quite divergent, the cytoplasmic portions of these proteins exhibit significant homology. The extracellular portions of TLR molecules contain leucine-rich repeat motifs (LRR) that confer pathogen/ligand interaction. Even though each individual receptor contains similar LRR motifs, each receptor is capable of recognizing structurally unrelated ligands. Within the cytoplasmic portion of the protein, IL-1Rs and TLRs all contain a block of ˜200 amino acids termed the toll/interleukin-1 receptor (TIR) domain. Regions within this domain are responsible for signal transduction via protein-protein interactions with other TIR containing proteins. The TLR family can be divided into 5 different subfamilies based on amino acid sequence similarities. (Table 1). The TLR2 subfamily encompasses TLR1, 2, 6 and 10 while members of the TLR9 subfamily include TLR7, 8 and 9. The recently identified murine TLR1 1 is closely related to TLR5. TLRs 3 and 4 are sufficiently distinct to remain separate subfamilies.

Other TLR family proteins, or homologs, orthologs, or proteins sharing at least about 50%, 60%, 70%, 80%, 90%, 95%, 97%, 99% or more of (nucleic acid and/or amino acid) sequence identity across different species may be readily obtained by, for example, sequence database searching using one of the TLRs described herein as query (e.g., BLAST search), or routine molecular biology techniques such as high/low stringency hybridization, antibody binding screen, etc. such TLR family proteins are also within the scope of the invention.

b. Expression.

The expression patterns of TLRs reflect their function. Hallman et al., Pediatr. Res. 50: 315-321, 2001. Most TLRs are expressed in cells of the immune system including dendritic cells, monocytes, peripheral blood lymphocytes and polymorphonuclear leukocytes. However, expression of some TLRs appears to be organ-specific. TLR11 for example, is found mainly in epithelial bladder and kidney cells where recognition of uropathogenic bacteria occurs. Zhang et al., Science 303: 1-522-1526, 2004. Other limited expression patterns include expression of TLR5 in ovary, testis and prostate, TLR2 expression in brain (Kurt-Jones et al., Proc. Natl. Acad. Sci. U.S.A. 101: 1315-1320, 2004), and TLR3 expression in placenta, pancreas and nervous system. Hallman et al., Pediatr. Res. 50: 315-321, 2001.

c. Function.

TLR members recognize both exogenous ligands (those expressed by invading microorganisms) and endogenous ligands that signal pathological danger (sterile inflammation) (reviewed in Zhang et al., Science 303: 1522-1526, 2004; Anders et al., J. Am. Soc. Nephrol. 15: 854-867, 2004). Pathogens recognized by the cell surface TLR2 subfamily members mainly consist of Gram-positive bacteria and heterodimerization of these receptors (TLR1+2, TLR2+6) can confer alternate ligand specificities. TLR2 also recognizes human cytomegalovirus (HCMV), herpes simplex virus (HSV) and measles virus. Kurt-Jones et al., Proc. Natl. Acad. Sci. U.S.A. 101: 1315-1320, 2004; Compton et al., J. Virol. 77: 4588-4596, 2003; Bieback et al., J. Virol. 76: 8729-8736, 2002. Endogenous ligands of TLR2 include oxygen radicals and components of necrotic cells. Anders et al., J. Am. Soc. Nephrol. 15: 854-867, 2004; Frantz et al., J. Biol. Chem. 276: 5197-5203, 2001. Ligands for TLR10 have yet to be identified. Members of the TLR9 subfamily are expressed intracellularly and recognize pathogen-specific nucleic acid motifs. Wagner, Trends Immunol. 25: 381-386, 2004. Most notable is the recognition of unmethylated CpG motifs by TLR9 and the detection of the anticancer agent, imiquimod, by TLR7. TLR5 recognizes bacterial flagella components. TLR11, the most recently identified murine TLR, recognizes components of uropathogenic bacteria but does not recognize non-pathogenic strains of Escherichia coli. Zhang et al., Science 303: 1522-1526, 2004. Although a sequence homologous to murine TLR11 has been identified in humans, the sequence contains multiple stop codons that would preclude expression of the protein. TLR3 is expressed intracellularly and recognizes dsRNA produced during infections. Takeda et al., Annu. Rev. Immunol. 21: 335-376, 2003. dsRNA recognition can be recapitulated through the use of synthesized nucleic acid “mimetics” such as polyinosine:polycytidylic acid (poly I:C). TLR3 and TLR9 may play redundant roles in recognition of viruses, as mice deficient in both (but not either one alone) are highly susceptible to infection with mouse cytomegalovirus. Tabeta et al., Proc. Natl. Acad. Sci. U.S.A. 101: 3516-3521, 2004. TLR4 was the first mammalian TLR-identified and is the most widely studied member. TLR4 recognizes lipopolysaccharide (LPS) produced by Gram-negative bacteria and is infamously known for inducing septic shock. TLR4 also responds to Taxol, respiratory synctial virus and Chlamydia pneumonia. Takeda et al., Annu. Rev. Immunol. 21: 335-376, 2003. Endogenous ligands for TLR4 may include extracellular matrix breakdown products (ex. fibrinogen, hyaluronic acid, heparan sulfate) and the host defense peptide, β-defensin.

d. TLR Signaling.

Binding of ligand to TLRs results in an ensuing signal transduction cascade and stimulation of new gene expression. Two major cascades are stimulated by TLRs, the MyD88-dependent and MyD88-independent pathways (reviewed in Zhang et al., Science 303: 1522-1526, 2004). Myeloid differentiation primary-response protein 88 (MyD88) is absolutely critical in TLR signaling that results in stimulation of pro-inflammatory molecules. MyD88 contains a TIR domain that is responsible for bridging the cytoplasmic domain of TLRs to other signaling molecules. The result of signaling through MyD88 is activation of the immunological NFκB pathway and expression of proteins involved in inflammation (eg. TNF-α and IL-1). With the exception of TLR3, all TLR molecules transmit signals through MyD88. However, MyD88-deficient mice are not completely inhibited in activating TLR pathways. The discovery of another TIR-containing protein TRIF (TIR-domain containing adaptor protein inducing IFN-β) resulted in the identification of a second TLR-signaling pathway. TRIF is essential for the induction of type 1 interferons after ligand binding to TLR3 and TLR4. Double knock-out of MyD88 and TRIF abrogates all known TLR signaling pathways, suggesting these two molecules are the essential upstream proteins.

III. Methods for Generating Non-Human Animal Models for Diabetic Complications

The present invention provides methods for producing non-human animal models for a diabetic complication. Such non-human animal models exhibit at least one (one or more) diabetic complication, which can be one or more microvascular complications, one or more macrovascular complications of one or more of each type of complication. In the methods, a non-human animal is administered a TLR agonist in an amount sufficient to induce a diabetic complication in the animal.

The non-human animal preferably is a rodent, such as a rat or a mouse. A non-human animal may also be another mammal, including, for example, a hamster, a guinea pig, a horse, a pig, a goat, a sheep, or other non-human primates. For ease of description, rat will be used as the model animal throughout the application to illustrate the invention.

In certain embodiments, rat models for type 1 diabetes are used. Rat models for type 1 diabetes include spontaneous diabetic strains and induced diabetic strains. Examples include BBDP/Wor, BBDR/Wor, LEW.1WR1 strains (for a list of the rat models for type 1 diabetes, see Mordes et al., ILAR Journal 45: 277-290, 2004). In certain other embodiments, rat models for type 2 diabetes are used. Such rats include, for example, BBZDR/Wor rats (see Tirabassi et al., ILAR Journal 45: 292-302, 2004). In certain further embodiments, any readily available inbred rat strains may be used with the methods of the invention to generate a rat model for a diabetic complication.

The term “TLR agonist” refers to an agent that potentiates the signaling activity of at least one TLR. A TLR agonist may act directly or indirectly on a TLR. An example of a direct TLR agonist is a TLR ligand, which may be a natural ligand or a synthetic ligand. Table 1 shows known TLR family members and their natural ligands and synthetic molecules known to activate them. See Ulevitch, Nature Review Immunology 4: 512-520, 2004. In certain preferred embodiments, a TLR agonist that activates one or more of TLR2, TLR3, TLR4, TLR7, TLR9 and TLR11 are used. An indirect TLR agonist may be a molecule that activates the TLR signaling by acting on a component upstream or downstream of TLR in the signaling pathway. Examples of a TLR signaling pathway component include, for example, MyD88, IRAK1, TRAF6 and NF-kB.

The TLR agonist may be given to the rats in a variety of ways, such as orally, topically, parenterally e.g. subcutaneously, intraperitoneally, by viral infection, or intravascularly

In certain embodiments, the methods of the invention produce rat models that have a (at least one, one or more) diabetic complication which occurs earlier and/or with greater severity than in currently available rat models, such as those described herein. In certain further embodiments, methods of the invention produce rat models in which the average time to develop a diabetic complication is 6 months, 5 months, 4 months, 3 months, 2 months, 1 month or less than the average time in which a currently available animal model (e.g., those described herein) develops corresponding or equivalent complications. In certain further embodiments, methods of the invention produce rat models in which the average time to develop a diabetic complication is about 7 months, 6 months, 5 months, 4 months, 3 months, 2 months, 1 month or less.

In certain preferred embodiments, the methods of the invention generate rat models that develop at least one complication in the absence of severe hyperglycemia.

A particular TLR may lead to one specific diabetic complication or lead to the induction of multiple diabetic complications. The invention also contemplates using an appropriate combination of TLR agonists to generate a rat model for one or more diabetic complications.

The diabetic complications include, but are not limited to, neuropathy, retinopathy, nephropathy, peripheral circulation disorders, erectile dysfunction in male diabetic patients, and skin ulcerations. These complications may be assessed using methods known to one of skill in the art. Examples of such methods are described in Ellis et al., Free Radic. Biol. Med. 24: 111-120, 1998; Ellis et al., Free Radic. Biol. Med. 28: 91-101, 2000; Ellis et al., Nitric. Oxide. 6: 295-304, 2002; Pierson et al., J. Neuropathol. Exp. Neurol. 61: 857-871, 2002; Sima et al., Diabetologia 43: 786-793, 2000; Sima et al., Diabetologia 44: 889-897, 2001; Xu and Sima, J. Neuropathol. Exp. Neurol. 60: 972-983, 2001; McVary et al., Am. J. Physiol. Regul. Integr. Comp. Physiol. 272: R259-R267, 1997. The exemplification section provides further examples of such assays.

TABLE 1
TLR Family Members

| | Exogenous Ligands^{a} | Endogenous Ligands^{b} | Synthetic Ligands |
|---|---|---|---|
| TLR1 | Triacetylated lipoproteins | | Triacyl |
| | | | lipopeptides |
| TLR2 | Zymosan; | Oxygen radicals; | Di-and triacyl |
| | Lipoproteins/lipopeptides; | Necrotic cells; HSP70 | lipopeptides |
| | Peptidoglycan; HCMV, | | |
| | HSV; Measles; lipoteichoic | | |
| | acid; lipoarabinomannan; | | |
| | Atypical lipopolysaccharide | | |
| TLR6 | Zymosan; Diacetylated | | Diacyl lipopeptides |
| | lipoproteins | | |
| TLR10 | ND | ND | ND |
| TLR3 | dsRNA; ssRNA; MCMV | | Poly I:C |
| TLR4 | LPS; Taxol; RSV | Hsp 60; ECM | Synthetic lipid A, |
| | | breakdown products; β- | E5564 |
| | | defensin 2; | |
| | | oligosaccharides of | |
| | | hyaluronic acid | |
| TLR5 | Baceterial flagellin | | Discontinuous 13- |
| | | | amino acid peptide |
| TLR11 | Uropathogenic bacteria | | |
| TLR7 | Viral RNA; Influenza | | Imidazole |
| | | | quinolines |
| | | | (imiquimod, |
| | | | resiquimod); |
| | | | Guanosine |
| | | | nucleotides |
| | | | (loxoribine); |
| | | | oligonucleotides |
| TLR8 | Viral RNA | | Imidazole |
| | | | quinolines |
| | | | (imiquimod) |
| TLR9 | Unmethylated CpG DNA; | CpG chromatin-IgG | CpG |
| | viral DNA; Bacterial DNA; | complexes | oligodeoxynucleotides |
| | MCMV, HSV | | |

^{a}HCMV—human cytomegalovirus; HSV—herpes simplex virus, RSV—respiratory syncytial virus; MCMV—mouse cytomegalovirus
^{b}ECM—Extracellular matrix
ND—not determined

IV. Screening Methods

The animal models created by the methods of the invention will enable screening of therapeutic agents useful for treating or preventing a diabetic complication. Accordingly, the present invention provides methods for identifying therapeutic agents for treating or preventing a diabetic complication. The methods comprise administering a candidate agent to an animal model made by the methods of the present invention, assessing at least one diabetic complication in the animal model as compared to a control animal model to which the candidate agent has not been administered. If at least one diabetic complication is reduced in symptoms or delayed in onset, the candidate agent is an agent for treating or preventing the diabetic complication.

The candidate agents used in the invention may be pharmacologic agents already known in the art or may be agents previously unknown to have any pharmacological activity. The agents may be naturally arising or designed in the laboratory. They may be isolated from microorganisms, animals, or plants, or may be produced recombinantly, or synthesized by chemical methods known in the art. They may be small molecules, nucleic acids, proteins, peptides or peptidomimetics. In certain embodiments, candidate agents are small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. In certain embodiments, the candidate agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. (Lam, Anticancer Drug Des. 12: 145, 1997).

Examples of methods for the synthesis of molecular libraries can be found in the art, for example in: DeWitt et al. Proc. Natl. Acad. Sci. U.S.A. 90: 6909, 1993 ; Erb et al. Proc. Natl. Acad. Sci. U.S.A. 91:11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; Kuruvilla et al., Nature 416: 653-657, 2002; and in Gallop et al., J. Med. Chem. 37: 1233, 1994.

In certain further embodiments, known pharmacological agents may be subjected to directed or random chemical modifications, such as acylation, alkylation, esterification, amidification, etc. to produce structural analogs.

The same methods for identifying therapeutic agents for treating a diabetic complication can also be used to validate lead compounds/agents generated from in vitro studies.

The candidate agent may be an agent that down regulates one or more TLR signaling pathway. In certain embodiments, the candidate agent may be a TLR antagonist.

V. Methods for Treating a Diabetic Complication

The present invention further provides methods for treating, inhibiting, relieving or reversing a diabetic complication. In the methods, an agent that interferes with a TLR signaling cascade is administered to an individual in need thereof, such as, but not limited to, diabetic individuals in whom such complications are not yet evident and those who already have at least one complication. In the former instance, such treatment is useful to prevent the occurrence of such complications (e.g., microvascular complications) and/or reduce the extent to which they occur. In the latter instance, such treatment is useful to reduce the extent to which such complications occur, prevent their further development or reverse the complications. In certain embodiments, the agent that interferes with a TLR signaling cascade may be an antibody specific for a TLR. In certain further embodiments, the agent that interferes with a TLR signaling cascade is selected from: a TLR2 antagonist, a TLR3 antagonist, TLR4 antagonist, a TLR5 antagonist, a TLR7 antagonist, a TLR9 antagonist, and a TLR11 antagonist.

Diabetic complications include retinopathy, neuropathy, nephropathy, peripheral circulation disorders, and skin ulcerations. An agent that interferes with a TLR signaling cascade may also prove effective in preventing, ameliorating, alleviating and gaining recovery from various symptoms and abnormalities caused by those diabetic complications, as exemplified by blindness, proteinurea, pain, numbness, psychroesthesia, intermittent claudication and gangrene.

One or more TLR antagonists may be administered with other therapeutic agents for treating diabetic complications. See, for example, US20030050301A1, entitled “Combination of aldose reductase inhibitors and angiotensin-II antagonists for the treatment of diabetic nephropathy”, and U.S. Pat. No. 6,218,411, entitled “Therapeutics for diabetic complications.”

The methods of the invention may also be applicable for treating a microvascular disease or condition that is not the result of diabetic complication. Generally, microvascular disease is a process through which the very small branches of arteries throughout the body become damaged. Microvascular disease may be a common component of other conditions, such as diabetes mellitus and autoimmune diseases. The most common symptoms are pain and discoloration of the extremities, usually the fingers and toes, sometimes even leading to gangrene. Microvascular disease usually affects the whole body to some degree and the most serious complications are caused by damage to the vital organs (e.g. heart, brain, kidneys, liver).

VI. Methods for Validating Therapeutic Agents of Diabetic Complications Using BBZDR/Wor Rats

As shown in more detail in the Exemplification section, Applicants have demonstrated that the diabetic obese male BBZDR/Wor rats show classic diabetes progression, including diabetic complications that are similar to those seen in human patients. Accordingly, the present invention further provides methods for validating lead compounds/agents generated from in vitro studies. In the methods, an obese male BBZDR/Wor rat is administered a lead compound for treating diabetic complications at various stage of its life, and maintained under conditions appropriate for diabetic rats. At appropriate time points, the rat is examined for one or more diabetic complications. If the rat shows reduced symptoms of a diabetic complication compared to a control rat that did not receive the lead compound, the lead compound is a validated compound for treating a diabetic complication.

The practice of aspects of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (Glover ed., 1985); Oligonucleotide Synthesis (Gait ed., 1984); Mullis et al. U.S. Pat. No: 4,683,195; Nucleic Acid Hybridization (Hames & Higgins eds., 1984); Transcription And Translation (Hames & Higgins eds., 1984); Culture Of Animal Cells (R. I. Freshney, Alan R. Liss, Inc., 1987); Immobilized Cells And Enzymes (IRL Press, 1986); Perbal, A Practical Guide To Molecular Cloning (1984); the treatise, Methods In Enzymology (Academic Press, Inc., N.Y.); Gene Transfer Vectors For Mammalian Cells (Miller and Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (Weir and Blackwell, eds., 1986); The Laboratory Rat, editor in chief: Mark A. Suckow; authors: Sharp and LaRegina. CRC Press, Boston. 1988.

All patents, patent applications and references cited herein are incorporated in their entirety by reference.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The methods and reagents described herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

It should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### EXEMPLIFICATION

The invention now being generally described, it will be more readily understood by reference to the following examples, which are included merely for purposes of illustration of certain embodiments and embodiments of the present invention, and are not intended to limit the invention.

Introduction

Diabetic Microvascular Complications

The results of the United Kingdom Prospective Diabetes Study and the Diabetes Control and Complications Trial (DCCT) have clearly shown that long-term glycemic control is important in preventing the accompanying complications of diabetes. Keen, Health Trends 26: 41-43, 1994; Klein, Diabetes Care 18: 258-268, 1995; Malone et al., Diabetes Care 24: 522-526, 2001; Molyneaux et al., Diabetes Res. Clin. Pract. 42: 77-83, 1998; Peterson and Smith, Am. Fam. Physician 52: 1092-1098, 1995; Service and O'Brien, Diabetologia 44: 1215-1220, 2001; Tirabassi et al., Diabetes 53(Suppl 2), 2004; Zinman, Clin. Cornerstone 1: 29-38, 1998; Zhang et al., Diabetes Care 24: 1275-1279, 2001.

Patients undergoing intensive insulin therapy developed diabetic complications at a much slower rate than patients receiving conventional insulin therapy. The consequences of repeated bouts of hyperglycemia without intensive insulin therapy results in altered flux through metabolic pathways that lead to the development of diabetic complications. Deckert et al., Diabetologia 14: 371-377, 1978; Deckert et al., Diabetologia 14: 363-370, 1978. The DCCT follow-up study, the Epidemiology of Diabetes Interventions and Complications (EDIC) study, highlighted another important aspect of the development of diabetic complications. White et al., J. Pediatr. 139: 804-812, 2001; JAMA 287: 2563-2569, 2002; N. Engl. J. Med. 342: 381-389, 2000. Due to the obvious benefit of intensive insulin therapy observed during the DCCT trial, patients formerly receiving conventional insulin therapy were switched to intensive insulin therapy regimens. Multiple year follow-ups demonstrated that these patients developed complications at a rate similar to those that never received additional intensive therapy. This suggests that during the development of diabetic complications, a pathological process is initiated as a direct result of high glucose concentrations. This process continues even when glucose levels are properly regulated by insulin therapy.

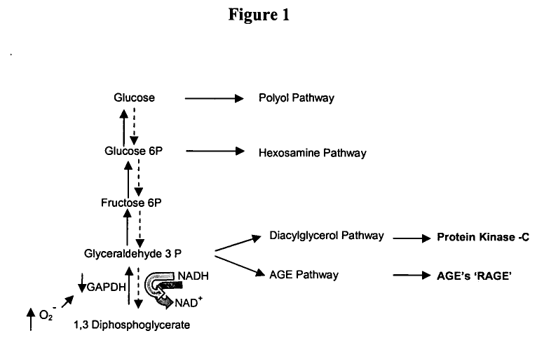

Microvascular disease refers to a group of disorders which result in diabetic neuropathy, retinopathy and nephropathy. These complications partially develop due to vascular damage of the supporting endothelium. Glucose-mediated vascular damage is directly caused by flux within four metabolic pathways. A unifying theme has been advanced to explain how the flux within these pathways is altered (FIG. 1). In this hypothesis, high levels of glucose result in overproduction of superoxides by the mitochondrial electron transport chain. Brownlee, Nature 414 : 813-820, 2001; Du et al., J. Clin. Invest. 112: 1049-1057, 2003; Hammes et al., Nat. Med. 9: 294-299, 2003. Increased production of superoxides decreases GAPDH activity which in turn leads to a build-up of intermediates in the glycolysis pathway. Excess intermediates are then shunted through four alternative pathways: the polyol pathway, resulting in increases in sorbitol and fructose and decreases in the threshold for intracellular oxygen injury; the diacylglycerol (DAG) pathway which activates protein kinase C (PKC); the hexosamine pathway which results in altered O linked glycosylation of proteins and alterations in signaling through serine and threonine phosphorylation; and the advanced glycation end products (AGE) pathway which results in direct damage to endothelial cells. Brownlee, Nature 414: 813-820, 2001; Du et al. (supra). The development of therapies to control and/or correct diabetic complications is directed at enzymes that control these pathways and restore the altered flux. Although this model explains how these pathways are collectively activated under high glucose conditions and the generation of O₂ radicals, it does not explain why these pathways cause pathological damage. In addition, this model only explains damage that occurs in cell types which cannot regulate intracellular concentrations of glucose (i.e. endothelial cells).

We recognize that the damage caused by these pathways and other physiological changes in diabetics may be initiated through ligation of toll-like receptors (TLRs). Further, we recognize that ligation of TLRs may initiate a self-perpetuating inflammatory process that is maintained even upon return to normal glucose homeostasis. Thus, the development of complications could be regulated by genetic components (TLR polymorphisms) and environmental factors (ligation of TLRs during infection).

Diabetic Neuropathy. Diabetic neuropathy is the most common diabetic complication, affecting 60-70% of all diabetic patients. Sima and Sugimoto, Diabetologia 42: 773-788, 1999. Diabetic neuropathy is a major complication that can present as several syndromes, which affect motor, sensory, and autonomic nerves. One of these syndromes, diabetic polyneuropathy (DPN), is a heterogeneous condition with symptoms ranging from peripheral sensory deficits (loss of sensation in limbs) to autonomic neuropathy. Levitt et al., Diabetes Care 19: 751-754, 1996; Tesfaye et al., Diabetologia 39: 329-335, 1996. Fulminant DPN can result in amputation of limbs and significant mortality once the autonomic nervous system is involved. Peripheral nerve dysfunction in DPN results from the additive effects of nerve damage accompanied by impaired nerve regeneration. Pierson et al., J. Neuropathol. Exp. Neurol. 61: 857-871, 2002; Sima, Cell Mol. Life Sci. 60: 2445-2464, 2003; Xu and Sima, J. Neuropathol. Exp. Neurol. 60: 972-983, 2001; Xu et al., J. Neuropathol. Exp. Neurol. 61: 164-175, 2002.

Diabetic Retinopathy. The frequency of diabetic retinopathy increases proportionally to the duration of diabetes and blood glucose control. Jiang et al., in Diabetes Mellitus: A Fundamental and Clinical Text, LeRoith, Taylor, Olefsky, Eds. (Lippencott-Raven, New York, 1996), chap. 80. Diabetic retinopathy can be divided into three clinical stages. Howard, in Diabetes Mellitus: A Fundamental and Clinical Text, LeRoith, Taylor, Olefsky, Eds. (Lippincott-Raven, New York, 1996). The first two stages, background retinopathy and preproliferative retinopathy, are characterized by microvascular abnormalities. Microaneurysms are the earliest clinically visible manifestation of background retinopathy, followed by retinal hemorrhages, focal leakage of proteins, and capillary nonperfusion that can lead to retinal edema. Additional microvascular abnormalities result from significant vascular occlusion and characterize the preproliferative retinopathy stage. These changes result in more severe retinal ischemia, including new blood vessels arising from the retina or optical disc, which define the third stage, proliferative retinopathy. Approximately 50% of patients who reach the preproliferative stage will progress to proliferative retinopathy within 15 months. Jiang et al., in Diabetes Mellitus: A Fundamental and Clinical Text, LeRoith, Taylor, Olefsky, Eds. (Lippencott-Raven, New York, 1996), chap. 80. Overgrowth of these vessels can lead to hemorrhage, retinal tears, and retinal detachment. Treatment options for diabetic retinopathy are limited to repeated laser surgery to stem new vessel growth. Aiello, Am. J. Ophthalmol. 136: 122-135, 2003.

Diabetic Nephropathy. Diabetic nephropathy is the leading cause of end-stage renal disease in the United States, and it affects both type 1 and type 2 diabetic patients. Not all individuals who develop diabetes progress to renal disease, which suggests a genetic component that predisposes some individuals to the development of diabetic nephropathy. Quinn et al., Diabetologia 39: 940-945, 1996. Structural changes in the diabetic kidney include overall increase in kidney size and glomerular volume, mesangial cell proliferation, accumulation of glomerular extracellular matrix, glomerular sclerosis, and tubular fibrosis. Osterby et al., APMIS 109: 751-761, 2001. The early stages of decreased function in the kidney result in increased urinary albumin excretion. Fulminant kidney disease in the diabetic is characterized by proteinuria, hypertension, and progressive renal failure. Trevisan and Viberti, in Diabetes Mellitus: A Fundamental and Clinical Text, LeRoith, Taylor, Olefsky, Eds. (Lippencott-Raven, New York, 2004). Diabetic nephropathy in humans is correlated with faulty activity of transforming growth factor-beta (TGF-β) family members, key regulators of cell proliferation, and the metabolism of extracellular matrix proteins (ECM; Huang et al., Diabetes 51: 3577-3581, 2002). In addition, upregulation of tissue transglutaminase (TGase) in diabetic patients leads to increased deposition of ECM. Skill et al., Lab Invest 81: 705-716, 2001. Overexpression and/or decreased breakdown of ECM proteins such as collagen lead to the basement membrane thickening that is characteristic of the early stages of nephropathy. Tsilibary, J. Pathol. 200: 537-546, 2003.

Animal Models to Study Diabetic Complications

The NOD Mouse. The Non-Obese Diabetic mouse (NOD) is a widely used model for studies of the pathogenesis of human autoimmune type I diabetes. In the NOD mouse, destruction of the pancreatic β-cells is a T cell mediated process leading to hyperglycemia among 80-90% of female and 20-40% of male mice. Beta cell destruction and hyperglycemia slowly progress and mice can survive without exogenous insulin therapy for 3-4 weeks after hyperglycemia is first detected. Atkinson and Leiter, Nat. Med. 5: 601-604, 1999. The NOD mouse is generally considered a less than ideal animal model for studies of type 1 diabetes and diabetic complications due to its body size, shortened lifespan, lack of ketoacidosis, deafness and absence of C5 complement. Atkinson and Leiter, Nat. Med. 5: 601-604, 1999. In addition, all mice, including the NOD, are lacking in aldose reductase activity and therefore do not accumulate sorbitol, a compound pivotal in the polyol pathway for diabetic complications. Yagihashi, Frontiers in Diabetic Research III: 459-463, 1990.

Streptozotocin induced diabetic rat: The antibiotic streptozotocin (STZ), isolated from Streptomycetes achromogenes, is used therapeutically to treat endocrine tumors. Oberg, Expert. Rev. Anticancer Ther. 3: 863-877, 2003. STZ treatment of rats leads to hyperglycemia due to its ability to selectively induce necrosis of insulin-secreting pancreatic β-cells (reviewed in Szkudelski, Physiol Res. 50: 537-546, 2001). STZ-induced hyperglycemia has served as a model of type 1 diabetes for almost 40 years and much of our understanding of neuronal impairment stems from these studies (reviewed in Gispen and Biessels, Trends Neurosci. 23: 542-549, 2000). Chemical ablation of islet cells is not complete however, and STZ-induced animals do not require insulin therapy for survival. Residual expression of insulin and C-peptide do not accurately approximate human disease and the STZ-induced diabetic rat is therefore limited for studies of diabetic complications.

Biobreeding (BB) Rats. The Biobreeding diabetes prone (BBDP/Wor), Biobreeding diabetes resistant (BBDR/Wor) and Biobreeding Zucker diabetic rats (BBZDR/Wor) are the best-characterized set of genetically similar animals for studying diabetes. Mordes et al, ILAR. J. 45: 278-291, 2004; Tirabassi et al., ILAR. J. 45: 292-302, 2004. The BBDP/Wor rat develops spontaneous autoimmune diabetes mellitus and is currently the best characterized rat model of human type 1 diabetes. Salient features include: genetic predisposition, abrupt onset of insulin dependent, ketosis-prone diabetes, and T-cell dependent autoimmune destruction of pancreatic β-cells. Mordes et al., ILAR. J. 45: 278-291, 2004; Mordes, in Frontiers in animal diabetes research, Primer on animal models of diabetes pp. 1-41, 2000. Since the first description of the syndrome in 1977, more than one thousand papers using this model have been published by laboratories throughout the world. Mordes, in Frontiers in animal diabetes research, Primer on animal models of diabetes pp. 1-41, 2000; Nakhooda et al., Diabetes 26: 100-112, 1977. BBDP/Wor rats develop more severe and more “human like” complications of diabetes than do NOD mice or animals treated with, streptozotocin or alloxan, to induce chemical diabetes.

See, for example, Sima and Sugimoto, Diabetologia 42: 773-788, 1999; Levitt et al., Diabetes Care 19: 751-754, 1996; Tesfaye et al., Diabetologia 39: 329-335, 1996; Pierson et al., J. Neuropathol. Exp. Neurol. 61: 857-871, 2002; Sima, Cell Mol. Life Sci. 60: 2445-2464, 2003; Xu and Sima, J. Neuropathol. Exp. Neurol. 60: 972-983, 2001; Xu et al., J. Neuropathol. Exp. Neurol. 61: 164-175, 2002; Jiang et al., in Diabetes Mellitus: A Fundamental and Clinical Text, LeRoith, Taylor, Olefsky, Eds. (Lippencott-Raven, New York, 1996), chap. 80.

This model also has limitations. All diabetes prone BBDP/Wor strains are severely lymphopenic from birth and experience a lifelong reduction of T-lymphocytes in peripheral blood, spleen and lymph nodes. Guberski et al., Diabetes 38: 887-893, 1989. Although widely used in studies of diabetic complications, the presence of lymphopenia with its associated susceptibility to many environmental pathogens limits the utility of lymphopenic BBDP/Wor rats in long-term studies of diabetic complications. The BBDR/Wor rat was derived from the BBDP/Wor rat in the fifth generation and is a nonlymphopenic rat strain that does not develop spontaneous diabetes. However, BBDR/Wor rats develop type 1 diabetes after environmental perturbation. Mordes et al., ILAR. J. 45: 278-291, 2004; Jun and Yoon, Diabetes Metab. Res. Rev. 19: 8-31, 2003; Yoon and Jun, ILAR. J. 45: 343-348, 2004.

We developed a new obese type 2 diabetic rat model with similar genetics to the BBDP/Wor and BBDR/Wor rat by introgressing the faulty Lepr^{fa} allele from Zucker fatty rats into the BB rat background. This model strain, BBZDR/Wor, is homozygous for the non-lymphopenia (LYP) gene (normal allele), shares the RT1^{u} MHC haplotype of lean BBDP/Wor and BBDR/Wor rats, and expresses the Lepr^{fa} (fa) fatty allele. Diabetes in the BBZDR/Wor rat exhibits sexual dimorphism, wherein >95% of the obese males but <3% of the obese females are hyperglycemic (Jun and Yoon, Diabetes Metab. Res. Rev. 19: 8-31, 2003). The recessive fa gene causes obesity, hypertension and insulin resistance; fa/fa homozygotes manifest obesity by the fourth week of life, and adults are considerably heavier than their lean heterozygous FA/fa littermates. Obese BBZDR/Wor rats exhibit beta cell hypersplasia and hyperinsulinemia, and diabetes is presumed to result from peripheral insulin resistance.

The BBDP/Wor and BBZDR/Wor rats have been used to study diabetic complications. Comparative diabetic neuropathy studies have highlighted important differences in the etiology of neuropathy in type 1 and type 2 diabetics. Sima et al., Diabetologia 43: 786-793, 2000. In both the type 1 and type 2 diabetic rats, DPN is characterized by a progressive slowing of nerve conduction velocity, axonal atrophy, and degeneration, however the slowing of nerve conduction velocities (the first sign of DPN in human and animal models) is more severe in BBDP/Wor (type1) rats than in BBZDR/Wor (type 2) rats. Furthermore, expression of early response genes required for nerve regeneration is delayed in BBDP/Wor rats but maintained at near-normal levels in BBZDR/Wor rats. Pierson et al., J. Neuropathol. Exp. Neurol. 61: 857-871, 2002. This difference may explain the increased efficiency of nerve regeneration seen in type 2 diabetic patients. In addition to developing neuropathy, BBZDR/Wor rats progress to late stages of preproliferative retinopathy. The initial steps involved in the development of diabetic retinopathy in these rats are well characterized and are similar to those seen in human patients. Finally, the BBZDR/Wor rat also develops duration-dependent nephropathy similar to that seen in human patients. Tirabassi et al., ILAR. J. 45: 292-302, 2004. Although these studies have shed important insight into the mechanisms behind diabetic complications, they have been hindered by the length of time 8-10 months required for rats to develop complications.

TLRs and Type 1 Diabetes

TLR3 ligation induces diabetes. Type 1 interferons are induced as a result of the anti-viral responses stimulated by dsRNA present during many viral infections. dsRNA binds to TLR3 to initiate the viral response pathway. Poly I:C is a dsRNA mimetic that was developed as an immunotherapeutic agent to induce interferons. High concentrations of poly I:C also induce autoimmune diabetes in several strains of inbred rats. Martin et al., Diabetes 48: 50-58, 1999; Ellerman and Like, Diabetologia 43: 890-898, 2000. Low doses of poly I:C alone do not induce hyperglycemia in BBDR/Wor rats. The induction of autoimmunity by TLRs may be a general mechanism. Darabi et al. have elegantly shown the role of TLRs in a mouse model of multiple sclerosis. Darabi et al., J. Immunol. 173: 92-99, 2004. Their results demonstrate that even in the presence of expanded autoreactive T cells, autoimmunity is only initiated upon microbial stimulation (TLR ligation) of immune cells.

Viral infection induces diabetes in autoimmune-prone-rats. Kilham's rat virus (KRV) has become the paradigm for viral induction of type 1 diabetes. In 1991, we demonstrated that infection of BBDR/Wor rats with KRV induced autoimmune diabetes in ˜30% of the rats without direct cytolytic infection of pancreatic β-cells. Guberski et al., Science 254: 1010-1013, 1991. The incidence of diabetes could be increased to nearly 100% by inclusion of the TLR3 liganad, poly I:C, just prior to viral infection. Ellerman et al., Diabetes 45: 557-562, 1996. However, poly I:C and KRV-induction of diabetes only occurs in young animals aged 20-25 days (see Preliminary Data). We have also recently demonstrated that intraperitoneal (i.p.) injection of 10⁴ plaque forming units (PFU) of RCMV in autoimmune-prone LEW.1WR1 rats induces diabetes in approximately 40% of the animals. Tirabassi et al., Rat Strain-Specific Susceptibility to Environmental Induction of Type 1 Diabetes. Diabetes 53(Suppl 2), 2004; Mordes et al., Autoimmune diabetes in the LEW.1WR1 rat after infection with Kilham rat virus (KRV) or rat cytomegalovirus (RCMV), Diabetes 52(Supplement), 2003. In contrast to KRV, injection of RCMV in BBDR/Wor rats does not induce diabetes. RCMV also accelerates the development of diabetes in spontaneous type 1 diabetic BBDP/Wor rats. Hillebrands et al., Clin. Dev. Immunol. 10: 133-139, 2003; van der et al., Clin. Dev. Immunol. 10: 153-160, 2003. Acceleration of diabetes does not appear to be due to direct infection of pancreatic β-cells but may be the result of changes in immune responses due to a viral-induced influx of macrophages. Combined with the poly I:C data, these results suggest that viruses may induce autoimmune disease by activation of toll signaling pathways. Indeed, the RCMV-homologous human and mouse cytomegaloviruses stimulate TLR2, TLR3 and TLR9. Compton et al., J. Virol. 77: 4588-4596, 2003. Tabeta et al., Proc. Natl. Acad. Sci. U.S.A. 101: 3516-3521, 2004. This assumption is further supported by the recent discovery that both KRV and poly I:C alter regulatory T cell balances. Chung et al., J. Immunol. 165: 2866-2876, 2000; Zipris et al., J. Immunol. 170: 3592-3602, 2003; Kobayashi et al., Clin. Exp. Immunol. 136: 423-431, 2004.

TLRs and Microvascular Complications

The newest advances in diabetic complications suggest that the inflammatory process contributes to progression of complications. Over all, serum markers of inflammation are significantly increased in type 2 diabetic patients and levels of the inflammatory marker, C-reactive protein are predictive of type 2 diabetes (reviewed in Crook, Diabet. Med. 21: 203-207, 2004). We recognize that TLR genetic polymorphisms and activation of different TLRs through exposure to endogenous ligands or micro-organisms may be responsible for accelerating or inhibiting disease progression. Based on this recognition, the TLR-regulated processes may be important targets for future therapeutics. These are described in further detail below.

Neuropathy. The essential clearance of cellular debris for subsequent nerve regeneration is altered in diabetic patients and may be a contributing factor to diabetic neuronal dysfunction. Elward and Gasque, Mol. Immunol. 40: 85-94, 2003. Inflammation, oxidative stress and apoptosis have been suggested as crucial mediators in diabetic neuropathy. This suggests that TLRs which recognize cellular breakdown products (TLR2 and TLR4) may be activated in diabetic central and peripheral nervous systems. Accordingly, a study evaluating the effects of common TLR4 polymorphisms on the development of diabetic neuropathy was recently undertaken. Rudofsky et al., Diabetes Care 27: 179-183, 2004. The Asp299Gly/Thr399Ile TLR4 genotypes decrease the level of response to TLR4 ligands and more importantly, reduce the prevalence of neuropathy in diabetic individuals. Activation of TLR4 has also been shown to be involved in exacerbation of neurodegeneration in a hypoxia-ischemia model. Lehnardt et al., Proc. Natl. Acad. Sci. U.S.A. 100: 8514-8519, 2003. Furthermore, a recent study has identified TLR2 as a key mediator in herpes simplex virus-(HSV) induced encephalitis. Kurt-Jones et al., Proc. Natl. Acad. Sci. U.S.A. 101: 1315-1320, 2004. Infection of TLR2 deficient mice with HSV showed that stimulation of TLR2 signaling results in central nervous system damage due to excessive inflammatory responses in the brain. Finally, TLR3 has also been implicated in inhibiting nerve regeneration. Cameron et al., Toll-like receptor 3 is a potent negative regulator of axonal growth in mammals, Toll 2004. Application of poly I:C to dorsal root explants and cultured neurons inhibits axonal outgrowth.

Retinopathy. Increased reactive oxygen species is a central feature of diabetic retinopathy (DR) and chronic, low-level inflammation is responsible for vascular lesions associated with DR. Sommeijer et al., Diabetes Care 27: 468-473, 2004. The stimulation of the inflammatory process by TLR2-recognition of oxygen radicals could be an important mechanism by which retinal damage occurs. Furthermore, the adenosine receptor signaling pathway may be an important mediator of hypoxic responses. Recent work has shown that this pathway synergizes with TLRs 2, 4, 7 and 9 to upregulate vascular endothelial growth factor (VEGF) expression, an initial step in angiogenesis. Leibovich et al., A synergistic interaction induced by adenosine A2A receptor agonists and TLR agonists mediates an angiogenic switch in macrophages, Toll 2004.

Nephropathy. Multiple TLRs may be involved in nephropathy. Anders et al., J. Am. Soc. Nephrol. 15: 854-867, 2004. For example, basement membrane thickening due to an excess of extracellular matrix (ECM) proteins is an early symptom of nephropathy. Chronic dysregulation of ECM deposition and breakdown could lead to signaling through TLR4. TLRs 3, 2/6 and 9, all recognize pathogens associated with kidney disease. Although not yet studied, TLR11 is also an ideal candidate as it is expressed in bladder and kidney.

In summary, we have described a novel mechanism for the development of diabetic complications. Pathological changes in diabetic patients result in the generation of molecules (O₂ radicals) and protein components that are known ligands for TLRs. These ligands stimulate self-perpetuating inflammatory processes through ligation of TLRs. Moreover, individual TLR polymorphisms and ligation of TLRs by microorganisms could further exacerbate the development of complications. This accounts for damage to all organs and encompasses the results obtained from the EDIC trial.

### Example 1

### BBZDR/Wor Rats

a. Obese Male BBZDR/Wor Rats but not Obese Female or Lean Littermates, Typically Develop Spontaneous Diabetes.

The mating scheme used to generate obese, type 2 diabetic rats results in three different genotype progeny. Littermates include lean (Fa/fa) male and female rats, obese (fa/fa) non-diabetic females and obese (fa/fa) diabetic males. We have observed that 98% of the obese males develop spontaneous diabetes by 85 days of age while obese female rats have impaired glucose tolerance (Table 2). These data highlight the value of this animal model. Studies can be performed with obese diabetic animals (males), obese nondiabetic animals (females) and lean nondiabetic littermates. With the exception of the Fa/fa allele, these animals are genetically identical: they express the same TLR alleles. Thus, the effects of TLR ligation on the development of complications can be assessed in normal glycemic and hyperglycemic littermates. This animal model trait is important to the studies outlined below.

TABLE 2
Incidence of Diabetes in BBRZDR/Wor Rats

| | BBZDR/Wor | | | |
|---|---|---|---|---|
| | Obese | | Lean | |
| | Male | Female | Male | Female |
| Incidence of | 98% | <3% | 0% | 0% |
| Diabetes | (N = 225) | (N = 217) | (N = 729) | (N = 783) |
| Age at Onset | 85 | >150 | N/A | N/A |
| (days) | | | | |

b. BBZDR/Wor Rats are Susceptible to Diabetes Triggered by Environmental Pertubation.

We have found that the BBZDR/Wor lean rats are susceptible to diabetes induced by KRV. During the initial development of the BBZDR/Wor strain, we performed several experiments to determine whether BBZDR/Wor animals are susceptible to other environmental triggers of diabetes. These results are compiled in Table 3. Lean and obese BBZDR/Wor rats were treated with either the TLR3 ligand, poly I:C, or poly I:C and KRV and the incidence of diabetes was determined. In these experiments, no animals treated with poly I:C alone developed diabetes. In contrast, 35-64% of animals treated with poly I:C+KRV became diabetic. We consistently observed a higher incidence of diabetes among lean BBZDR/Wor animals as compared to obese rats. More experiments will need to be performed to determine whether these differences are statistically significant. We recently revisited the poly I:C experiments in the now, fully-inbred (>40 generations) BBZDR/Wor rat line. In a preliminary experiment, lean and obese female animals were injected with the TLR3 ligand, poly I:C (5 μg/gm body weight, 3× a week) and were observed for the onset of diabetes. In contrast to our previous results, we observed that 40% of the lean animals and 50% of the obese females developed hyperglycemia. These results may contradict our previous observations due to the inbred status of the strain, or lot differences between poly I:C preparations. However, these data suggest that multiple environmental pertubants can induce diabetes in BBZDR/Wor lean and obese rats.

In summary, we have developed a type 2 diabetic rat strain, the BBZDR/Wor rat, which has proven useful in the study of diabetic complications. Littermates from this strain express three different phenotypes: lean, normal males and females, obese females with impaired glucose tolerance and obese diabetic males. Lean and obese animals are both affected by TLR ligation and environmental pertubation as evidenced by the development of diabetes after treatment with the TLR3 ligand, poly I:C, and infection with KRV.

TABLE 3
Environmental Induction of Diabetes

% Diabetic With Treatment

| BBZDR/Wor | Poly I:C | KRV + Poly I:C |
|---|---|---|
| Lean | 0% | 64% |
| Obese | 0% | 35% |

### Example 2

### BBZDR/Wor Rat Demonstrates Classic Diabetic Progression

The BBZDR/Wor rat is an inbred rat strain (>20 generations) that was developed as an animal model for type 2 diabetes and is emerging as the most applicable model of type 2 diabetic complications. To produce the BBZDR/Wor type 2 diabetic rat, classical genetic methods were used to remove the recessive Iddm2 gene responsible for lymphopenia and spontaneous autoimmunity and retain the Lepr^{fa} (fa¹) mutation by crossing BBZDP/Wor animals with the lean, nondiabetic BBDR/Wor rats. Both male and female obese BBZDR/Wor rats are infertile, and strain lines are maintained through mating of heterozygous (lean) littermates. Although obese females rarely (<1%) develop disease, the obese male BBZDR/Wor rat spontaneously develops type 2 diabetes at approximately 12 wk of age (>98%) when fed standard rat chow (Purina 5010; Ellis et al., Free Radic. Biol. Med. 24: 111-120, 1998; Ellis et al, Free Radic. Biol. Med. 28: 91-101; Ellis et al., Nitric Oxide 6: 295-304, 2002. Heterozygous lean rats of either sex do not develop glycosuria or hyperglycemia. Thus, obese females and lean littermates are generally used as age-matched controls, and except where noted otherwise, this article focuses on the research generated using the type 2 diabetic male.

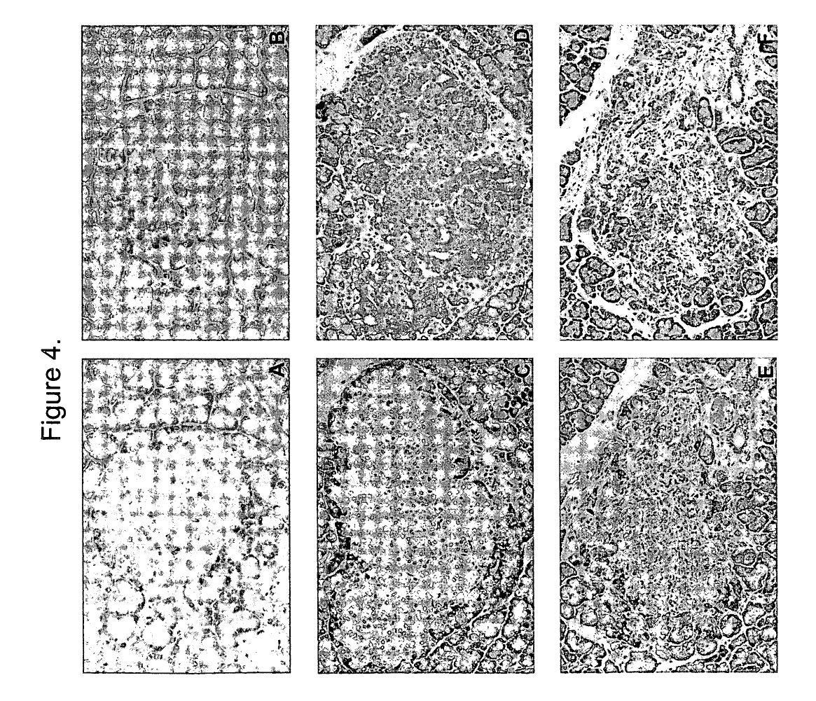

Salient features of the BBZDR/Wor diabetic rat include dyslipidemia, hyperglycemia, insulin resistance, hypertension, and decreased levels of the beta cell-specific glucose transporter type-2 (GLUT-2¹) (Ellis et al., Free Radic. Biol. Med. 24: 111-120, 1998; Sima and Merry, Exp. Clin. Endocrinol. Diabetes 105: 63-64, 1997. In comparison, we have observed that obese female BBZDR/Wor rats have impaired glucose tolerance, but rarely (<1%, age of onset 21 wk) develop hyperglycemia. In addition, as in human type 2 diabetics, we observed that the BBZDR/Wor male and female rats developed IGT (glycemic values that exceeded 200 mg/dL by 2 hr after dextrose injection) and insulin resistance. We analyzed pancreatic islets isolated from lean and obese BBZDR/Wor. Islets from lean nondiabetic BBZDR/Wor rats were normal in all respects and displayed maintenance of normal islet architecture and normal levels of glucagon, insulin, and GLUT-2 (FIGS. 4A and 4B; GLUT-2 not shown). In contrast, islets from young obese diabetic males were profoundly enlarged and demonstrated beta-cell hyperplasia and mild fibrosis, commonly seen in islets from patients with type 2 diabetes (FIGS. 4C and 4D). An overall reduction in insulin and GLUT-2 was also observed. Progression of diabetes led to a decrease in beta-cell mass and disorganization of islet architecture seen in older diabetic obese males (FIGS. 4E and 4F). Studies of pancreatic sections from older diabetic rats immunostained for insulin revealed focal reduction in beta-cell insulin content (FIG. 4F). We also observed an overall reduction of GLUT-2 staining of beta-cell surface membranes.

Collectively, these data show that similar to the human disease, the BBZDR/Wor type 2 rat demonstrates classic disease progression, as outlined below. Furthermore, rats with 4 mo of diabetes develop both microvascular and macrovascular complications, as described below.

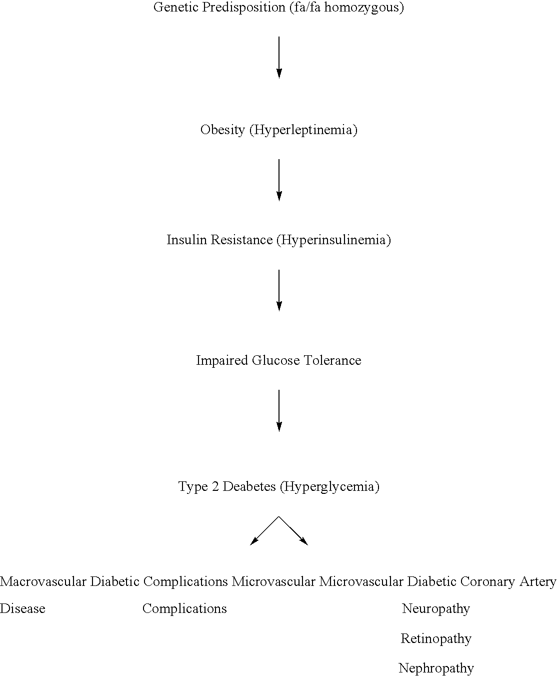

### Example 3

### Generation of New Rat Models for Diabetic Complications

a. Generation of Animals for Study

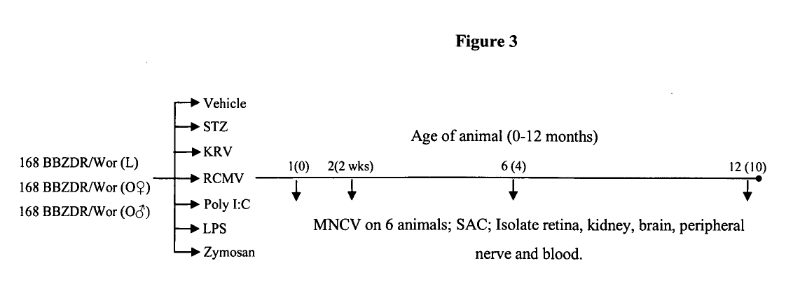

The roles of TLRs in diabetic animals are evaluated in two ways: First, the expression of TLRs in target organs in pre-diabetic, acutely diabetic and chronically diabetic animals is analyzed (Vehicle treated, FIG. 3). Second, whether TLR ligation has a positive or negative effect on diabetic complications is tested by comparing animals treated with 1) streptozotocin to induce hyperglycemia in the absence of TLR ligation, 2) infected with viruses that trigger potentially multiple TLRs, and, 3) administered agonists specific for particular TLRs. For all of these experiments, the lean, obese female and obese male diabetic BBZDR/Wor animals are used. This model system is extremely valuable in these studies. The three different phenotypic animals are littermates: with the exception of fatty alleles and sex chromosomes, these animals are genetically identical (i.e., TLR alleles are identical). Any differences observed between these animals can be attributed to the effects of dyslipidemia (obese not diabetic) and hyperglycemia (obese diabetic) superimposed on stimulation of TLRs.

b. Treatment of Animals

Lean male and female, obese female and obese male BBZDR/Wor rats greater than 35 days of age are treated as indicated with the agents listed in Table 4 (6 animals from each group per collection timepoint; total 168 lean male and female, 168 obese female and 168 obese male). Animals of this age are used in order to prevent the induction of autoimmune diabetes observed in weanling animals (20-25 days) after TLR ligation. Animals treated 3 times/week receive injections for 3 weeks or until the onset of diabetes. Control animals are injected with vehicle. To induce hyperglycemia in the absence of inflammation stimulated by TLR ligation, some animals are injected with streptozotocin (STZ).

TABLE 4
Treatment of Animals with TLR Agonists

| | Stimulated | |
|---|---|---|
| Agent | TLR | Regimen |
| Vehicle | None | 3 times/week |
| Streptozotocin | None | 30-65 mg/kg; 1 × |
| KRV | Unknown | 1 × 10⁷; 1 × |
| RCMV^{a} | TLRs 2, 3, 9^{b} | 2 × 10⁵; 1 × |
| Poly I:C | TLR3 | 5 μg/gm body weight; 3 times/ |
| | | week |
| Lipopolysaccharide | TLR4 | 100 μg/gm body wt; 3 times/week |
| Zymosan | TLR2 | 150 μg/gm body wt; 3 times/week |

STZ is a chemical agent which causes selective destruction of pancreatic beta-cells. Animals treated with this agent allow us to gauge the contribution of hyperglycemia versus hyperglycemia+TLR ligation in the progression of complications. Infection with KRV and RCMV has the potential to trigger multiple TLRs while additional animals are treated with specific TLR agonists. Obese males should develop diabetes spontaneously at approximately 85 days of age. Although animals are treated at an age where autoimmunity should not develop, all animals are carefully monitored for the development of ketosis-prone, autoimmune type 1 diabetes. Onset of diabetes is monitored 3 times weekly by testing for glycosuria and high blood glucose, as described in the General Methods. Following the onset of diabetes, animals are monitored closely for the development of ketonuria to determine whether they require exogenous insulin therapy. If required, diabetic animals are to be treated with daily-titrated doses of insulin to maintain blood glucose levels at ˜20 mmol/L (360 mg/dL; see General Methods). This level of hyperglycemia will lead to development of diabetic complications but will keep the animals from developing ketoacidosis. Level of glycemic control is confirmed at sacrifice by using blood samples to measure glycated hemoglobin content.

c. Evaluate the Effect of RCMV in BBZDR/Wor Rats.

Rat cytomegalovirus (RCMV) is a beta-herpesvirus that induces diabetes in the autoimmune-prone LEW.1WR1 rat-strain. Tirabassi et al., Diabetes 53(Suppl 2), 2004; Mordes et al., Diabetes 52(Supplement), 2003. RCMV also accelerates diabetes in the BBDP/Wor strain. Hillebrands et al., Clin. Dev. Immunol. 10: 133-139, 2003; van der et al., Clin. Dev. Immunol. 10: 153-160, 2003. Homologous human and mouse cytomegaloviruses stimulate TLRs 2, 3 and 9. Compton et al., J. Virol. 77: 4588-4596, 2003; Tabeta et al., Proc. Natl. Acad. Sci. U.S.A. 101: 3516-3521, 2004. A pilot study is performed in BBZDR/Wor rats to determine whether they are susceptible to RCMV infection. BBZDR/Wor lean male and female, BBZDR/Wor obese female, and BBZDR/Wor obese prediabetic males (4 of each) are infected by intraperitoneal injection of RCMV (2×10⁵ plaque forming units). Two control animals from each group are injected with vehicle for a total of 6 animals from each group per timepoint (18 animals total from each group). At 3, 8 and 14 days post infection, 4 infected and 2 uninfected animals from each group are sacrificed and livers, spleens and salivary glands are removed. The tissues are homogenized and assayed for infectious virus by titration on tissue culture cells. RCMV replicates first in the liver and spleen followed by replication in salivary glands. Once it is demonstrated that the BBZDR/Wor animals support replication of RCMV, RCMV is used as another TLR-agonist.

d. Compare TLR Expression in Retina, Kidney, Brain and Blood in Lean, Obese and Obese Diabetic Rats.

Animals treated with vehicle are removed from the study according to the following schedule: 1 month of age (prediabetic), 2 weeks post diabetes onset (acutely diabetic) and 4 and 10 months post diabetes onset (chronic diabetic). These time points are chosen based on observable pathological changes described in the literature. Pierson et al., J. Neuropathol. Exp. Neurol. 61: 857-871, 2002; Tirabassi et al., ILAR. J. 45: 292-302, 2004; Sima et al., Diabetologia 43: 786-793, 2000; Sima and Merry, Exp. Clin. Endocrinol. Diabetes 105: 63-64, 1997; Murray et al., Diabetes 45: 272, 1996. At each time point, 6 animals of each group undergo MNCV testing, as described below. The animals are then sacrificed and whole organs are removed and preserved in formalin. Samples from 4 of the animals are analyzed immunohistochemically using antibodies specific for TLR2, 3, 4, 6, 7, 9, and 10 on serial sections. Immunohistochemistry is performed on all samples at once to allow direct comparison over time. Whole cardiac blood is also obtained at each time point. Blood is used for FACs analysis using the specified TLR antibodies.

The immunohistochemsitry and FACs analysis provides us with a quick survey of TLR expression in the prediabetic, acutely diabetic and chronic diabetic animal. Increased expression of certain TLR family members in target organs as diabetic complications progress would support our hypothesis. If a great increase in expression of certain TLRs is seen as diabetes progresses, the time course is extended for an additional two months. Furthermore, if changes in particular TLRs are observed, expression of the TLR in animals treated with TLR agonists is analyzed.

e. Assess Progression of Neuropathy, Retinopathy and Nephropathy after Ligation of TLRs

To test whether TLR ligation has a positive or negative effect on diabetic complications, the progression of complications in animals treated with different TLR agonists is assessed and compared to animals treated with vehicle alone. These studies are performed using the group of animals described in FIG. 3.

Neuropathy. One of the first observable changes in diabetic nerves is the slowing of motor nerve conduction velocities (MNCV). To assess the progression of neuropathy, sciatic-tibial MNCV testing will be performed on anesthetized animals treated at each of the indicated time points, as described previously. Pierson et al., J. Neuropathol. Exp. Neurol. 61: 857-871, 2002. To obtain meaningful data, MNCV testing is performed on 6 animals from each group. Briefly, the left sciatic-tibial motor conduction system is stimulated proximally at the sciatic notch and distally at the ankle via bipolar electrodes with supramaximal stimuli. The latencies of the compound muscle action potentials are recorded vial bipolar electrodes from the first interosseous muscle and measured from the stimulus artifact to onset of the M-wave deflection. Each measurement is made between 8 and 16 times. MNCV is calculated by dividing the distance between the stimulating and recording electrode by the difference between the distal and proximal latencies. MNCV results from animals treated with vehicle or each of the TLR agonists are compared.

Retinopathy. Vascular endothelial growth factor (VEGF) expression is upregulated in both human and rat diabetic retinas. VEGF upregulation may be responsible for endothelial leakage. Immunohistochemistry on whole retinas is performed to assay VEGF expression as an indication of retinopathy progression. We compare retinas from 6 animals for each time point from animals treated with either vehicle or each of the agonists listed in Table 4. Immunohistochemistry is performed after all samples have been collected.

Nephropathy. Kidneys of diabetic rats display the presence of both nodular and diffuse glomerulosclerosis and increased glomerular size with concomitant mesangial and basement membrane thickening. Tirabassi et al., ILAR. J. 45: 292-302, 2004. Moreover, the BBZDR/Wor rat displays endothelial cell proliferation, interstitial fibrosis, and arteriolosclerosis. Disease severity correlates with duration of diabetes. Collectively, these changes are similar to those reported in human diabetic type 2 patients. Osterby et al., APMIS 109: 751-761, 2001. Furthermore, all diabetic animals show a 3- to 4-fold increase in collagen expression compared with nondiabetic animals. Increases in the collagen-positive area of the kidney proportionally correlate with the duration of diabetes, suggesting expansion of ECM components. Nephropathy is assessed in vehicle- and TLR-agonist-treated rats by analyzing kidney sections reacted with antibodies specific for collagen. The expansion of collagen expression is looked for as an indicator of kidney disease.

Data Analysis. The differences between groups are tested for statistical significance using ANOVA, one way means of variance. A P value less than 0.05 indicates statistical significance.

General Methods

Husbandry & Rat Strains: The BRM vivarium houses the BBZDR/Wor rat strain. All animals are maintained under strict barrier conditions and have been virus antibody free (VAF) since 1989. Acidified water and Purina Chow #5010 will be provided ad libitum. Rats will be exposed to a 12-hr. light/dark light cycle. All animals will be maintained in accordance with standards established by the National Research Council and local IACUC regulations.

Treatment of animals. Lean, obese female and obese male rats greater than 30 days of age will be injected intraperitoneally with one of the following agents: 1) Vehicle (saline), 2) 1 injection of 50 mg/kg streptozotocin, 3) 1 injection of 1×10⁷ plaque forming units of KRV, 4) 1 injection of 2×10⁵ plaque forming units of RCMV, 5) 5 μg/gm body weight poly I:C (Sigma-Aldrich, St. Louis, Mo.) 6) 100 μg/gm body weight LPS (Sigma-Aldrich, St. Louis, Mo.) 7) 150 μg/gm body weight zymosan (Sigma-Aldrich, St. Louis, Mo.).

Detection of diabetes: Prior to onset of diabetes, animals will be tested for diabetes 3× weekly. Diabetes is defined as the presence of glycosuria and blood glucose greater than 250 mg/dL (13.9 mmol/L). Monitoring will be performed by testing urine samples for glucose (Clinistix; Bayer Corp, Ind.). Animals with a positive glycosuria will be bled by nicking the tail and 50 μl of blood will be collected to measure serum glucose levels (Analox-GL5, Leominster, Mass.). Animals with diabetes will be studied twice weekly to determine blood glucose, urinary glucose and ketones (Clinistix and Ketostix, Bayer Corp., Elkhart, Ind.). Animals will be provided with exogenous therapy if ketonuria is present. If required, they will receive daily-titrated doses of protamine zinc insulin (PZI) to maintain blood glucose levels at ˜20 mmol/L (360 mg/dL). Since hypoglycemia has been reported to induce CNS neuronal loss, we will adjust our normal therapies by reducing the dose of insulin from 0.9 U/100 g body weight to 0.75 U/100 g body weight. This level of hyperglycemia will lead to development of diabetic complications but will keep the animals from developing ketoacidosis. Animals that have ketonuria will have their insulin therapy increased and will receive 20 mls of a solution comprised of 2 mls of 8.4% sodium bicarbonate dissolved in 18 mls of lactated ringers. Following rehydration, the insulin dose will be adjusted to maintain the rats in a moderate state of glycosuria in the absence of ketones. Hypoglycemia, as indicated by the presence of aglycosuria will result in a 15-25% decrease in the insulin dose and an injection of lactated ringers containing 1 ml of 50% dextrose. In severe hypoglycemia, as indicated by posturing and a blood glucose <40 mg/dL, an intraperitoneal injection of 1 mL of 50% dextrose will be administered. Throughout the study, body weights will be taken three times weekly and all animals will be observed daily by a technician and weekly by a veterinarian. Upon sacrifice, cardiac blood will be taken to determine the percent of glycated hemoglobin (HbA1c; A1c Now Monitor; Metrika). HbA1c is a measure of glycemic control over the prior four months (normal values ˜5%).

Flow cytometry. Briefly, 1×10⁶ viable cells will be first incubated with primary antibodies for 30 min on ice. Cells will be washed and incubated for an additional 30 min on ice with FITC-conjugated, PE-conjugated, or CyChrome®-conjugated antibodies. FITC-, biotin-, PE-, and CyChrome®-conjugated isotype control immunoglobulins will be used for all analyses. Cells will be washed, fixed with 1% paraformaldyhde, and analyzed using a FACScan® instrument (Becton Dickinson, Sunnyvale, Calif.). TLR specific antibodies will be purchased from Santa Cruz Biotechnologies (Santa Cruz, Calif.), antibody specific for rat collagen will be purchased from Research Diagnostics (Flanders, N.J.) and rat VEGF antibody will be purchased from R&D Systems (Minneapolis, Minn.). Secondary antibodies will be purchased from BD Pharmingen (San Diego, Calif.) and Jackson Immunoresearch Labs. Inc. (West Grove, Pa.).

Preparation of salivary gland passaged virus: Rat cytomegalovirus strain Maasticht will be isolated from the salivary glands of infected animals. Briefly, irradiated LEW.1WR1 rats will be infected I.P. with >1×10⁶ PFU of virus. Four weeks post infection, the animals will be sacrificed and the salivary glands will be removed. Homogenized tissue will be centrifuged and the supernatant containing virus will be aliquoted and stored until use. Viral titers will be determined by plaque assay on rat embryo fibroblasts.

### EQUIVALENTS

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

What is claimed is:

1. A method of providing a non-human animal model for at least one diabetic complication, the method comprising administering to the non-human animal a Toll-Like Receptor (TLR) agonist in an amount sufficient to induce said at least one diabetic complication in the animal.

2. The method of claim 1, wherein the TLR agonist is an agonist for TLR3.

3. The method of claim 1, wherein the animal is a rodent.

4. The method of claim 3, wherein the rodent is a rat.

5. The method of claim 4, wherein the rat is a biobreeding Zucker diabetic rat (BBZDR/Wor).

6. The method of claim 1, wherein the diabetic complication manifests in said animal at least about a month earlier than that in an available rat model selected from: Streptozotocin-induced diabetic rat, biobreeding diabetes prone rat (BBDP/Wor), biobreeding diabetes resistant rat (BBDR/Wor) or biobreeding Zucker diabetic rat (BBZDR/Wor).

7. The method of claim 1, wherein the diabetic complication manifests in said animal at least about 3 months earlier than that in an available rat model selected from: Streptozotocin-induced diabetic rat, biobreeding diabetes prone rat (BBDP/Wor), biobreeding diabetes resistant rat (BBDR/Wor) or biobreeding Zucker diabetic rat (BBZDR/Wor).

8. The method of claim 1, wherein the diabetic complication manifests in said animal at about 3 months after the administration of the TLR agonist.

9. The method of claim 6, wherein the diabetic complication is a microvascular complication or a macrovascular complication.

10. The method of claim 9, wherein the microvascular complication is neuropathy, retinopathy or nephropathy.

11. The method of claim 10, wherein the TLR agonist is an agonist for TLR2, TLR3, TLR4, TLR7, TLR9, or TLR11.

12. The method of claim 1, wherein the animal model develops said at least one diabetic complication in the absence of severe hyperglycemia and/or glycosuria.

13. The method of claim 1, comprising administering to the non-human animal two or more Toll-Like Receptor (TLR) agonists.

14. A method of screening for a therapeutic agent useful for treating or preventing a diabetic complication, comprising:
(a) providing, by the method of claim 1, a test animal and a substantially identical control animal;
(b) administering a candidate agent to the test animal;
(c) maintaining the test animal and the control animal under conditions appropriate for development of at least one diabetic complication in the control animal;
(d) assessing said at least one diabetic complication in the test animal and the control animal; and,
(e) comparing the severity and/or onset of the diabetic complication in the test animal with that of the control animal,
wherein reduced severity and/or delay in the onset of the diabetic complication in the test animal indicates that the candidate agent is the therapeutic agent useful for treating or preventing the diabetic complication.

15. The method of claim 14, wherein the animal is a rat.

16. The method of claim 15, wherein the rat is a BBZDR/Wor rat.

17. The method of claim 14, wherein the test animal and the control animal are littermates.

18. The method of claim 14, wherein the candidate agent is a TLR antagonist.

19. A method for treating, preventing, reversing or limiting the severity of a diabetic complication in an individual in need thereof, comprising administering to the individual an agent that interferes with TLR signaling, in an amount sufficient to interfere with TLR signaling.

20. A method of treating, preventing, reversing or limiting the severity of a diabetic complication in an individual in need thereof, comprising administering to the individual an agent that interferes with at least one TLR signaling cascade.
